(19) United States
(12) Patent Application Publication
(10) Pub. No.: US 2009/0176965 A1
(43) Pub. Date: Jul. 9, 2009

(54) Sericin Having Improved Antioxidant and Tyrosinase Inhibitive Abilities by Irradiation, and Methods of Making and Using the Same

(75) Inventors: Ju-Woon Lee, Jeollabuk-Do (KR); Jong-Il Choi, Seoul (KR); Jae-Hun Kim, Jeollabuk-Do (KR); Beom-Seok Song, Seoul (KR); Myung-Woo Byun, Jeollabuk-Do (KR); Eui-Baek Byun, Jeollabuk-Do (KR); Nak-Yun Sung, Chungcheongnam-do (KR)

Correspondence Address: STERNE, KESSLER, GOLDSTEIN & FOX P.L.L.C., 1100 NEW YORK AVENUE, N.W., WASHINGTON, DC, 20005, US

(73) Assignee: Korean Atomic Energy Research Institute, Daejeon (KR)

(21) Appl. No.: 12/324,162

(22) Filed: Nov. 26, 2008

(30) Foreign Application Priority Data
Jan. 4, 2008 (KR) 10-2008-0001230

(51) Int. Cl. C07K 7/08; C07K 1/00

(52) U.S. Cl. 530/326; 530/333

(57) ABSTRACT

Disclosed are sericin having improved antioxidant and tyrosinase inhibitory abilities and increased molecular weight by irradiation, which causes a modification of a sericin molecular structure, a preparation method thereof and use of the irradiated sericin in various applications including food products, cosmetics and/or pharmaceutical products and medicines to improve antioxidant ability and/or tyrosinase inhibitory functions.

### CROSS REFERENCE TO RELATED APPLICATIONS

This application claims priority to Korean Patent Application No. 10-2008-0001230, filed on Jan. 4, 2008, in the Korean Intellectual Property Office, the disclosure of which is incorporated herein by reference in its entirety.

### BACKGROUND OF THE INVENTION

1. Field of the Invention

The present invention relates to sericin having improved antioxidant and tyrosinase inhibitory abilities by irradiation, preparation thereof and use of the same, more particularly, to sericin having improved antioxidant and tyrosinase inhibitory abilities and increased molecular weight by irradiation causing modification of molecular structure, a preparation method thereof and use of the irradiated sericin in various applications including food products, cosmetics and/or pharmaceutical products and medicines to improve antioxidant ability and/or tyrosinase inhibitory functions.

2. Background Art

It is well known that sericin is one of polymeric proteins comprising eighteen (18) amino acids and having a wide range of molecular weight from 10 kDa to 300 kDa (see Wei, T., et al. “Preparation and structure of porous silk sericin materials,” Macromolecular Materials and Engineering 290:188-194 (2005)).

When sericin as a water soluble protein is dissolved in polar solvents, hydrolyzed by acid or alkaline solutions and/or decomposed by proteases, the size of sericin molecules is altered by different factors such as temperature, pH, processing time, etc.

High molecular weight sericin peptides with molecular weight of more than 20 kDa have been used as biomedical materials, functional membranes, hydrogels and/or functional fibers (see A. Ogawa, et al., J. Biosci. Bioeng. 98:217 (2004)).

Since regeneration of sericin which was generally produced from silk proteins can achieve remarkable economic development and social benefits, there is a strong requirement for techniques and processes to regenerate sericin as described above.

### BRIEF SUMMARY OF THE INVENTION

Accordingly, in studying sericin with improved physiological activities, the present inventors found and suggested that molecular structure of unmodified or native sericin can be modified by irradiating a sericin solution to produce high molecular weight sericin having improved radical scavenging ability and tyrosinase inhibitory ability, therefore, thereby completing the present invention.

An object of the present invention is to provide sericin having improved physiological activities by irradiation causing modification of molecular structure thereof.

Another object of the present invention is to provide a method for preparation of sericin having improved physiological activities by irradiation causing modification of molecular structure thereof.

Still a further object of the present invention is to provide a use of sericin having improved physiological activities by irradiation causing modification of molecular structure thereof.

In order to accomplish the above described objects, the present invention provides sericin having improved antioxidant and tyrosinase inhibitory abilities and increased molecular weight by irradiation causing modification of molecular structure thereof.

Also, the present invention provides a method for preparing a sericin having improved antioxidant and tyrosinase inhibitory abilities and an increased molecular weight by modifying a molecular structure of the sericin by irradiating the sericin to deliver an absorption dose of radiation to sericin in the range of about 10 kiloGray (“kGy”) to about 500 kGy.

Additionally, the present invention provides use of sericin with modified molecular structure to manufacture a variety of products for improvement of antioxidant and tyrosinase inhibitory abilities including, for example, food products, cosmetics and/or pharmaceutical products and medicines.

As the high molecular weight sericin having molecular structure modified by irradiation, in particular, gamma(γ)-ray irradiation according to the present invention represents excellent biological characteristics such as improved radical removing ability, tyrosinase inhibitory effect, etc., as well as a whitening effect, compared to conventional sericin materials as controls, the present inventive sericin is useful for production of food products, cosmetics and/or pharmaceutical products and medicines.

### BRIEF DESCRIPTION OF THE DRAWINGS/FIGURES

These and other objects, features, aspects, and advantages of the present invention will be more fully described in the following detailed description of embodiments and examples, taken in conjunction with the accompanying drawings. In the drawings:

FIG. 1 is a graph showing results observed by UV absorption spectrum for sericin protein obtained by gamma(γ)-ray irradiation;

FIG. 2 is a histogram showing results of secondary structure analysis of sericin observed by far-UV CD spectrum for sericin protein obtained by γ-ray irradiation;

FIG. 3 shows results of molecular weight of sericin protein measured by GPC for sericin protein obtained by γ-ray irradiation;

FIG. 4 is a histogram showing results of improved DPPH radical scavenging ability of sericin protein obtained by γ-ray irradiation; and

FIG. 5 is a histogram showing results of improved tyrosinase inhibitory ability of sericin protein obtained by γ-ray irradiation.

Reference will now be made in detail to the embodiments of the present invention, examples of which are illustrated in the accompanying drawings.

### DETAILED DESCRIPTION OF THE INVENTION

This specification discloses one or more embodiments that incorporate the features of this invention. The disclosed embodiment(s) merely exemplify the invention. The scope of the invention is not limited to the disclosed embodiment(s). The invention is defined by the claims appended hereto.

The embodiment(s) described, and references in the specification to “one embodiment,” “an embodiment,” “an example embodiment,” etc., indicate that the embodiment(s) described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is understood that it is within the knowledge of one skilled in the art to effect such feature, structure, or characteristic in connection with other embodiments whether or not explicitly described.

References to spatial descriptions (e.g., “above,” “below,” “up,” “down,” “top,” “bottom,” etc.) made herein are for purposes of description and illustration only, and should be interpreted as non-limiting upon the methods of the present invention, and sericin prepared therefrom, which can be spatially arranged in any orientation or manner.

An aspect of the present invention in order to accomplish the above objects is to provide sericin having improved antioxidant and tyrosinase inhibitory abilities and increased molecular weight by irradiation causing modification of molecular structure thereof. Thus, the present invention is directed to irradiated sericin having improved antioxidant and tyrosinase inhibitory abilities, an increased molecular weight, and/or a modified molecular structure compared to a sericin that has not been irradiated.

Sericin used in the present invention is obtainable from, inter alia, silkworm cocoons. More particularly, silkworm cocoons were treated with an aqueous sodium carbonate solution. The treated cocoons were heated and filtered to prepare a sericin solution. From the sericin solution, impurities were removed through general purification processes such as dialysis, etc. The sericin solution after removal of impurities can be further processed into a powdery state for use by lyophilizing the solution.

The radiation used in the irradiation can be selected from: gamma(γ)-ray, electron beam, X-ray radiation, and combinations thereof. In some embodiments, the radiation is gamma(γ)-ray or electron beam, in view of molecular weight increasing effect of sericin after the irradiation.

With regard to radiation, absorption dose of irradiation can be about 10 kGy to about 500 kGy, about 50 kGy to about 300 kGy, or about 50 kGy to about 200 kGy. With the absorption dose of irradiation of less than 10 kGy, a desirable irradiation effect is not represented and, if the absorption dose of irradiation is more than 500 kGy, there can be caused a problem such as decomposition of ingredients in sericin by high irradiation dose.

For sericin having modified molecular structure by irradiation according to the present, it was observed by UV absorption spectrum that absorbance of the present inventive sericin was increased more than 2 times at 280 nm, and by more than 10 times at 300 nm, respectively, compared to sericin without irradiation as a control (i.e., unmodified sericin).

The modification of molecular structure of sericin according to the present invention can be generally classified into a decrease of alpha(α)-helix secondary structure and an increase of secondary structure, wherein the increased secondary structure is selected from: a beta(β)-sheet, a β-turn, a random coil, and combinations thereof.

Although the modification of molecular structure of sericin is the decrease of α-helix secondary structure or the increase of at least one secondary structure selected from a γ-sheet, a γ-turn, a random coil, and combinations thereof, in some embodiments the modification includes all of a decrease of α-helix secondary structure and an increase of each of the secondary structures of β-sheet, β-turn and random coil, in view of maximum improvement of antioxidant and tyrosinase inhibitory abilities.

Lee et al. reported that the covalent bonds of a protein in a solution phase are can be broken by oxygen radicals generated by irradiation and molecular structure of the protein is collapsed, thereby resulting in modification of secondary and tertiary structures of the protein, in Lee, S. et al., “Effect of gamma-irradiation on the physicochemical properties of porcine and bovine blood plasma proteins,” Food Chem. 82:521 (2003), the entire contents of which are herein incorporated by reference.

A β-turn structure comprising four (4) residual groups can be formed by a hydrogen bond between an i-th carbonyl group and a third after i-th (i-th+third) amine group, and is a typical element that produces a spherical form of a protein and is commonly represented on a surface of the spherical protein. A β-turn structure can promote structural folding by reversing the direction of polypeptide chains. Therefore, β-turn structure is a very important element in structural folding of natural proteins.

The sericin having modified molecular structure by irradiation according to the present invention can have molecular weight ranging from about 2 kDa to about 1000 kDa, about 2 kDa to about 500 kDa, or about 8 kDa to about 135 kDa. As described above, while sericin without irradiation has molecular weight of not more than 2 kDa, the present inventive sericin can have considerably increased molecular weight as a result of irradiation.

The sericin having modified molecular structure by irradiation according to the present invention exhibits improved radical removing ability of at least about 3 times or more, at least about 4 times or more, or at least about 5 times or more compared to typical sericin without irradiation, thereby improving physiological activities such as antioxidant ability.

Furthermore, the sericin having modified molecular structure by irradiation according to the present invention exhibits improved tyrosinase inhibitory effect of at least about 3 times or more, at least about 4 times or more, or at least about 5 times or more compared to typical sericin without irradiation, thereby providing a sericin having an improved whitening effect.

It is generally known that melanin pigment in skin of a human is an important pigment mechanism to protect UV based damage, but abnormal pigment formation by melanin such as melasma, freckles, senile lentigines, excessive pigment, etc. can cause undesirable problems. It is known in the related art that tyrosinase causes biosynthesis of melanin in skin of a human being and tyrosinase inhibitory agents or chemicals are important materials for manufacturing whitening cosmetics.

Another aspect of the present invention is to provide a method for preparing a sericin having an improved antioxidant and/or tyrosinase inhibitory ability. In some embodiments, the sericin also has an increased molecular weight by induced by irradiation of the sericin. In some embodiments, the improved antioxidant and/or tyrosinase inhibitory ability and/or increased molecular weight can be induced by irradiation of sericin to deliver an absorption dose of radiation to sericin in the range of about 10 kGy to about 500 kGy.

Sericin used in the present inventive method for preparation of sericin by irradiation is sericin extracted from silkworm cocoons or artificially synthesized sericin. The sericin to be used is extracted by treating the silkworm cocoons in an aqueous sodium carbonate solution, heating and filtering the treated solution, and removing impurities from the solution through dialysis or the like. In some embodiments, a powder form of sericin is used by lyophilizing the purified sericin solution after removal of impurities.

On the other hand, the synthesized sericin can include sericin prepared by biosynthesis using microorganisms and/or by polypeptide synthesis method commonly available in the related art.

In some embodiments, the method for preparing an irradiated sericin according to the present invention further includes lyophilizing the sericin after removal of impurities, which can be carried out using conventionally known processes.

The radiation used in the irradiation method of the present invention can be selected from: gamma(γ)-ray, electron beam, X-ray radiation, and combinations thereof.

In some embodiments, the radiation is gamma(γ)-ray or electron beam, in view of molecular weight increasing effect of sericin after the irradiation.

The modification of molecular structure of sericin according to the present invention can be generally classified into decrease of alpha(α)-helix secondary structure and increase of secondary structure of at least one selected from: a beta(β)-sheet, a β-turn, a random coil, and combinations thereof.

Although the modification of molecular structure of sericin is the decrease of α-helix secondary structure or the increase of secondary structure of at least one selected from a β-sheet, a β-turn, a random coil, in some embodiments the modification includes all of the decrease of a-helix secondary structure and the increase of each of secondary structures of β-sheet, β-turn and random coil, in view of maximum improvement of antioxidant and tyrosinase inhibitory abilities.

The sericin having modified molecular structure by irradiation according to the present invention can have a molecular weight of about 2 kDa to about 1000 kDa, about 2 kDa to about 500 kDa, or about 8 kDa to about 135 kDa.

A further aspect of the present invention is to provide application of sericin having modified molecular structure obtained by the present invention in manufacturing a variety of products for improvement of antioxidant and tyrosinase inhibitory abilities, which include food products, cosmetics and/or pharmaceutical products and medicines.

The application of sericin according to the present invention for production of food products, cosmetics and/or pharmaceutical products and medicines, can be varied according to requirements within a constant range acceptable by food codes, food additive codes, designation and standards for cosmetic raw materials, regulations in regard to test procedures, etc.

Examples of food products using the present inventive sericin with modified molecular structure include beverages, noodles, frozen foods, dairy products, meat processing products, food products with special properties, seasoning foods, extraction processed products, uncooked foods, etc., but are not limited thereto.

Examples of cosmetics using the present inventive sericin with modified molecular structure include formulations such as lotion, cream, gel, etc., but are not limited thereto.

Examples of pharmaceutical products and medicines include formulations such as tablet, granule, pill, liquid, injections, cream, ointment, etc., but are not limited thereto.

Methods for manufacturing food products, cosmetics or pharmaceutical products and/or medicines are not particularly restricted but include general processes commonly available in the related art.

Hereinafter, the present invention will be more particularly described by the following examples. However, these are intended to illustrate the invention in its various embodiments and do not limit the scope of the present invention.

### EXAMPLE 1

### Preparation of Sericin having Modified Molecular Structure by γ-Ray Irradiation

Sericin raw material used in the present invention was natural sericin extracted from silkworm cocoons. 10 g of silkworm cocoons was treated using 200 ml of an aqueous sodium carbonate solution with 5% by weight per volume (“w/v”), heated for 1 hour, and filtered using a filter paper to remove dissolved sericin fraction from the solution. Using hot water, the residue was washed several times to remove sericin residue and sodium carbonate. The purified solution underwent dialysis to remove sodium carbonate then lyophilization to prepare sericin powder as a test sample.

The prepared sericin powder sample was treated by irradiation using a cobalt-60 irradiator in Advanced Radiation Technology Institute, Korea Atomic Energy Research Institute (Jeongup, Korea). An irradiation source had a capacity of about 300 kCi and an irradiation dose rate of 10 kGy per hour.

The irradiation dose rate was determined using a 5 mm diameter alanine dosimeter (Bruker Instruments, Rheinstetten, Germany). A dosimetry system was used after standardization in compliance with IAEA standards.

After dissolving silk sericin in distilled water to result in a solution at a concentration of 1 mg/mL, the solution was treated using Co-60 γ-ray irradiation equipment (IR-79, Nordion International Ltd., Ontario, Canada) with an irradiation dose rate of 10 kGy per hour to reach a total absorption dose of irradiation of 5 kGy, thereby producing a sericin solution with modified molecular structure.

### EXAMPLE 2

### Preparation of Sericin having Modified Molecular Structure by γ-Ray Irradiation

A sericin solution having modified molecular structure was prepared by the same procedure described in Example 1, except that γ-ray irradiation was carried out to reach a total absorption dose of irradiation of 10 kGy.

### EXAMPLE 3

### Preparation of Sericin having Modified Molecular Structure by γ-Ray Irradiation

A sericin solution having modified molecular structure was prepared by the same procedure described in Example 1, except that γ-ray irradiation was carried out to reach a total absorption dose of irradiation of 50 kGy.

### EXAMPLE 4

### Preparation of Sericin having Modified Molecular Structure by γ-Ray Irradiation

A sericin solution having modified molecular structure was prepared by the same procedure described in Example 1, except that γ-ray irradiation was carried out to reach a total absorption dose of irradiation of 100 kGy.

### EXAMPLE 5

### Preparation of Sericin having Modified Molecular Structure by γ-Ray Irradiation

A sericin solution having modified molecular structure was prepared by the same procedure described in Example 1, except that γ-ray irradiation was carried out to reach a total absorption dose of irradiation of 150 kGy.

### EXAMPLE 6

### Preparation of Sericin having Modified Molecular Structure by γ-Ray Irradiation

A sericin solution having modified molecular structure was prepared by the same procedure described in Example 1, except that γ-ray irradiation was carried out to reach a total absorption dose of irradiation of 200 kGy.

### EXPERIMENTAL EXAMPLE 1

### UV Spectrum Analysis

The γ-ray irradiated silk sericin solutions prepared in Examples 1 to 6, were used in this experimental example while storing the solutions at 4° C.

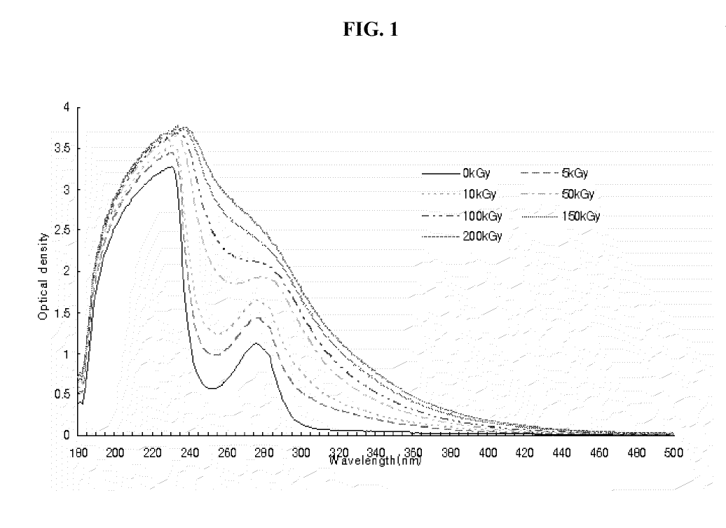

In order to examine structural modification of silk sericin by γ-ray irradiation, after dissolving each of the silk sericin solutions at a concentration of 2 mg/ml and γ-ray irradiating the solution, the irradiated solution was subjected to UV-VIS spectrum analysis at 180 nm to 500 nm using a UV spectrophotometer (UV-1601 PC, Shimadzu Corp., Tokyo, Japan). The analysis result is shown in FIG. 1. In this case, sericin without γ-ray irradiation was used as a control.

From UV absorption spectrum, structural modification was observed by absorbance of branch chains of aromatic amino acid on surface of protein. Three kinds of amino acids such as phenylalanine, tyrosine and tryptophan generally have aromatic branch chains and absorb light in UV regions of UV absorption spectrum, which are similar to most of compounds having bond rings.

Tyrosine and tryptophan mostly have UV absorbance at 280 nm, absorption rate of tryptophan is 100 times stronger than that of phenylalanine, and UV absorbance of phenylalanine is mostly measured at 206 nm.

Referring to FIG. 1, absorbance of γ-ray irradiated silk sericin is higher at 260 nm and 280 nm as irradiation dose is increased. Variation of UV absorbance indicates structural modification caused by irradiation. Such structural modification resulted because internal amino acids such as tryptophan and tyrosine were externally exposed by division of protein structure. It was found that turbidity was higher at 330 nm as irradiation dose was increased.

From results of reported studies, maximum absorption wavelength region was 214 nm. This indicated that peptide bonds are groups mostly absorbing sericin in UV region and the above results were supported by this Experimental Example 1.

### EXPERIMENTAL EXAMPLE 2

### Circular Dichroism Spectrum Analysis

Circular dichroism spectrum (hereinafter, referred to as “CD spectrum”) was measured using a Jasco J-715 spectropolarimeter (Japan Spectroscopic) equipped with a 150 W xenon lamp.

Far-UV spectrum was measured at 190 nm to 250 nm. A sample (0.2 mg/mL) was analyzed in PBS solution at pH 7.2 using a 1 mm cuvette after washing the sample with nitrogen gas.

Analysis was repeated three times and mean value of the analysis results was calculated after subtraction of measured value for PBS, wherein unit of CD spectrum analysis result was represented by residual ellipticity (degree cm²/dmol).

The absorption difference between left-handed polarized light and right-handed polarized light generated by structural unbalance was determined by CD spectroscopy, which can measure secondary structure of protein at far-UV spectral region of 190 to 250 nm. Chromophor at this region is protein bond and, when such bonds are positioned in regular folded environment, α-helix, β-sheet and/or random structures, different CD spectra with specific patterns and dimensions appear.

Two negative peaks exposed at 208 nm and 220 nm, respectively, are known to demonstrate a protein having α-helix secondary structure while a peak at 214 nm expresses a protein having β-sheet secondary structure.

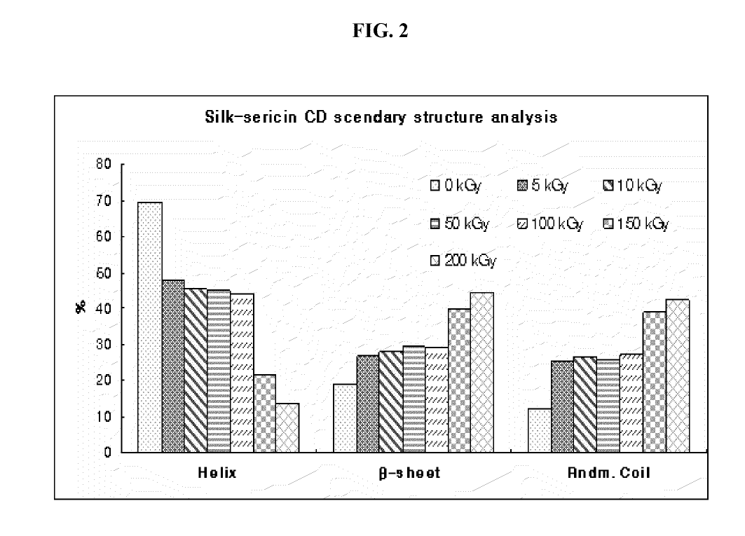

The secondary structure of sericin is modified by irradiation. Referring to FIG. 2, it was demonstrated that α-helix secondary structure is decreased as irradiating dosage is increased, while β-sheet, β-turn and random coil structures are increased in relation to decrease of α-helix secondary structure.

### EXPERIMENTAL EXAMPLE 3

### Molecular Weight Analysis Using Gel Permeation Chromatography (GPC)

Molecular weight of γ-ray irradiated sericin was determined by gel permeation chromatography (GPC)-high performance liquid chromatography (HPLC).

As a HPLC system, Waters Alliance HPLC system (Mo. 2690, MA, USA) together with PL aquagel-OH column (300×7.5 mm, 8 μm; Polymer Laboratories, Ltd. UK) was adopted.

0.1 M sodium nitrate solution as a mobile phase was flowed through the column at a flow rate of 1 mL/min for 40 minutes. Pullulan standard for GPC was purchased and available from Showa Denko Co.

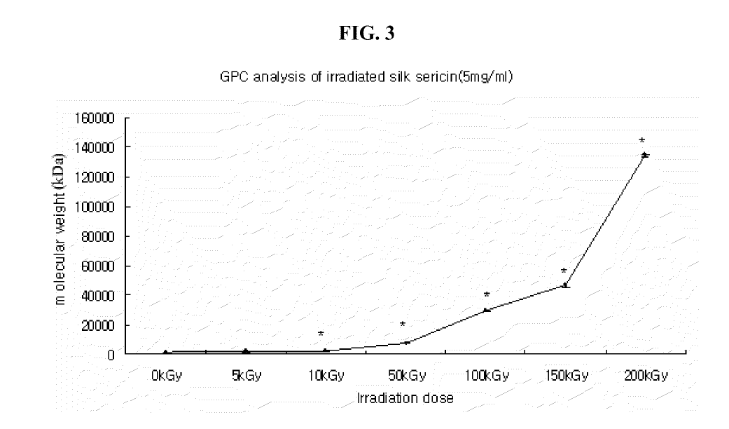

FIG. 3 shows variation of molecular weight of silk sericin under different irradiation doses to represent effect of irradiation. Referring to FIG. 3, it was found that molecular weight of silk sericin without irradiation was less than 6 kDa and silk sericin irradiated with an irradiation dose ranging from 5 kGy to 10 kGy had similar molecular weights to that of the sericin without irradiation.

However, silk sericin irradiated with an irradiation dose of more than 10 kGy, more particularly, 50 kGy, 100 kGy, 150 kGy and 200 kGy had molecular weights of 8 kDa, 30 kDa, 47 kDa and 135 kDa, respectively. Such results indicated that intermolecular combination is increased by structural modification as the irradiation dose is increased.

### EXPERIMENTAL EXAMPLE 4

### Determination of Radical Removing Ability of Sericin having Modified Molecular Structure by γ-Ray Irradiation

Electron donating ability for each of sericin samples prepared in Examples 1 to 6 was determined according to Blois method which measures hydrogen donation effect of silk sericin to DPPH (2.2-diphenyl-1-picryl-hydrazil).

To 2 mL of each of the sericin samples at constant concentration, 2 mL of 1×10⁻⁴ M DPPH solution in 99% ethanol was added and the mixture was reacted at 37° C. for 30 minutes during vortex mixing. The reaction product was subjected to measurement of absorbance at 517 nm. For the electron donation effect, difference of absorbance before and after adding the sericin sample was represented by percent (%).

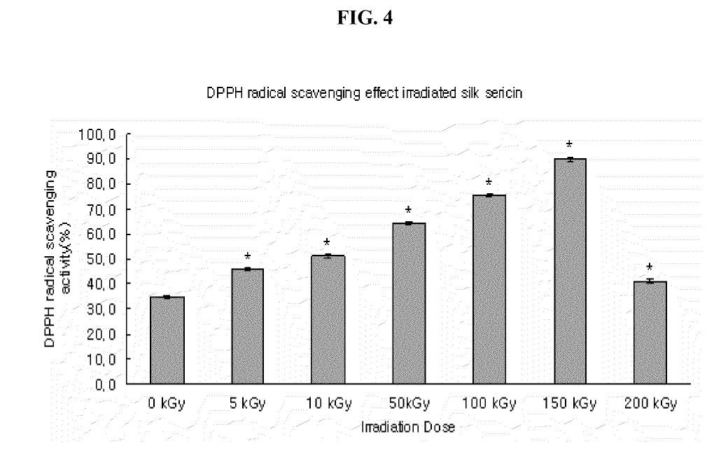

DPPH as a stable free group with absorbance at 517 nm was used to study radical removing ability of silk sericin. FIG. 4 shows antioxidant effect of irradiated silk sericin. Referring to FIG. 4, it was found that DPPH radical removing ability of irradiated silk sericin was higher than that of silk sericin at 0 kGy when both of the silk sericins have the same concentration, and antioxidant ability was improved as the irradiation dose was increased.

### EXPERIMENTAL EXAMPLE 5

### Determination of Tyrosinase Inhibitory Effect of Sericin having Modified Molecular Structure by γ-Ray Irradiation

Tyrosinase inhibitory effect for each of sericin samples prepared in Examples 1 to 6 was determined in order to examine whitening activity of silk sericin by γ-ray irradiation.

A substrate solution was prepared by dissolving 10 mM L-DOPA (L-3,4-dihydroxyphenylalanine; Sigma Chemical Co., St. Louis, Mo., USA) in 0.5 mL of 0.175 M sodium phosphate buffer at pH 6.8. 0.2 mL of the substrate solution was mixed with 0.1 mL of a sample solution and 0.2 mL of mushroom tyrosinase (100 U/mL, Sigma USA) was added thereto. After reacting the mixture at 25° C. for 15 minutes, the reaction product, that is, DOPA chromium was subjected to measurement of absorbance at 475 nm. For tyrosinase inhibitory activity, absorbance reducing rate of the reaction product with addition of the sample solution was represented by percent (%) relative to a control without addition of the sample solution.

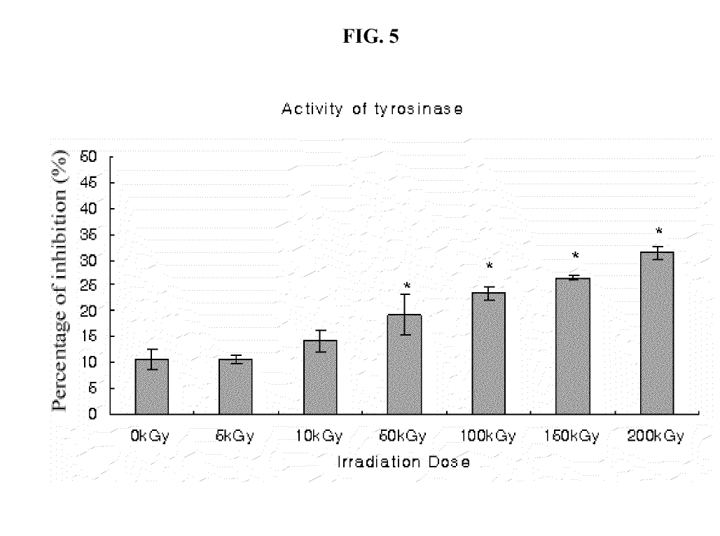

FIG. 5 shows tyrosinase inhibitory effect of sericin according to the present invention. Referring to FIG. 5, it was observed that all of γ-ray irradiated silk sericins expressed tyrosinase inhibitory effect higher than that of silk sericin without irradiation and tyrosinase inhibitory effect was improved as the irradiation dose was increased.

From the above experimental results, it was demonstrated that silk sericin obtained by irradiation has excellent effect of inhibiting tyrosinase activity and the irradiated sericin exhibits higher antioxidant effect compared to sericin without irradiation.

As the high molecular weight sericin having molecular structure modified by irradiation, in particular, gamma(γ)-ray irradiation according to the present invention represents excellent biological characteristics such as improved radical removing ability, tyrosinase inhibitory effect, etc. as well as whitening effect, compared to conventional sericin materials as controls, the present inventive sericin is useful for production of food products, cosmetics and/or pharmaceutical products and medicines.

### CONCLUSION

These examples illustrate possible embodiments of the present invention. While various embodiments of the present invention have been described above, it should be understood that they have been presented by way of example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the following claims and their equivalents.

All documents cited herein, including journal articles or abstracts, published or corresponding U.S. or foreign patent applications, issued or foreign patents, or any other documents, are each entirely incorporated by reference herein, including all data, tables, figures, and text presented in the cited documents.

What is claimed is:

1. Irradiated sericin having improved antioxidant and tyrosinase inhibitory abilities, an increased molecular weight, and a modified molecular structure compared to a sericin that has not been irradiated.

2. The irradiated sericin according to claim 1, wherein the sericin is irradiated with a radiation selected from: gamma(γ)-ray, electron beam, X-ray, and combinations thereof.

3. The irradiated sericin according to claim 1, wherein the sericin is irradiated with an absorption dose of about 10 kGy to about 500 kGy of a radiation.

4. The irradiated sericin according to claim 1, wherein the modified molecular structure comprises a decrease in an alpha(α)-helix secondary structure.

5. The irradiated sericin according to claim 1, wherein the modified molecular structure comprises an increase in a secondary structure selected from: a beta(β)-sheet, β-turn, a random coil, and combinations thereof.

6. The irradiated sericin according to claim 1, wherein the modified molecular structure comprises a decrease in an α-helix secondary structure and an increase in a secondary structure selected from: a β-sheet, a β-turn, a random coil, and combinations thereof.

7. The irradiated sericin according to claim 1, wherein the increased molecular weight is about 2 kDa to about 1,000 kDa.

8. A method for preparing sericin having improved antioxidant and tyrosinase inhibitory abilities and an increased molecular weight, the method comprising modifying a molecular structure of sericin by irradiating sericin with a radiation at an absorption dose of about 10 kGy to about 500 kGy of the radiation.

9. The method according to claim 8, wherein the sericin to be irradiated was extracted from silkworm cocoons or artificially synthesized.

10. The method according to claim 8, further comprising lyophilizing the sericin after the irradiating.

11. The method according to claim 8, wherein the radiation used in the irradiating is selected from: gamma(γ)-ray, electron beam, X-ray, and combinations thereof.

12. The method according to claim 8, wherein the modification of molecular structure comprises decrease of alpha(α)-helix secondary structure.

13. The method according to claim 8, wherein the modifying the molecular structure of sericin comprises increasing a secondary structure of the sericin selected from: a beta(β)-sheet, a β-turn, a random coil, and combinations thereof.

14. The method according to claim 8, wherein the modifying the molecular structure of sericin comprises decreasing an α-helix secondary structure of the sericin and increasing a secondary structure of the sericin selected from: a beta(β)-sheet, a β-turn, a random coil, and combinations thereof.

15. The method according to claim 8, wherein the sericin has a molecular weight ranging of about 2 kDa to about 1000 kDa.

16. A sericin product prepared by the process of claim 8.

17. The sericin product of claim 16, wherein the sericin product is selected from: a food product, a cosmetic product, a pharmaceutical product, and combinations thereof.

18. A food product comprising the irradiated sericin of claim 1, wherein the food product exhibits improved antioxidant and tyrosinase inhibitory abilities compared to a food product comprising sericin that is not irradiated.

19. A cosmetic product comprising the irradiated sericin of claim 1, wherein the cosmetic product exhibits improved antioxidant and tyrosinase inhibitory abilities compared to a cosmetic product comprising sericin that is not irradiated.

20. A pharmaceutical product comprising the irradiated sericin of claim 1, wherein the pharmaceutical product exhibits improved antioxidant and tyrosinase inhibitory abilities compared to a pharmaceutical product comprising sericin that is not irradiated.
